(19) United States
(12) Patent Application Publication
(10) Pub. No.: US 2012/0128616 A1
(43) Pub. Date: May 24, 2012

(54) COSMETIC COMPOSITION COMPRISING AT LEAST ONE ELASTOMERIC POLYURETHANE AND AT LEAST ONE CATIONIC POLYMER

(76) Inventors: Sébastien VOISIN, Levallois Perret (FR); Laëtitia Feuillette, Paris (FR)

(21) Appl. No.: 12/950,076

(22) Filed: Nov. 19, 2010

(51) Int. Cl. A61K 8/87; A61Q 5/06; A61Q 5/00; A61K 8/89; A61K 8/92

(52) U.S. Cl. 424/70.9; 424/70.17; 424/70.12

(57) ABSTRACT

Provided is a cosmetic composition comprising, in a cosmetically acceptable medium, at least one film-forming elastomeric, anionic polyurethane, and at least one cationic polymer, as well as a method for the styling of the hair comprising applying this composition to the hair.

Provided is a cosmetic composition comprising, in a cosmetically acceptable medium, at least one film-forming polymer with certain elastomeric characteristics and at least one cationic polymer.

Also provided is a method of styling the hair comprising applying the cosmetic composition to the hair.

Fixing of the hairstyle is an important element of styling, and involves the maintenance of shape. The term “styling composition” refers to any type of hair-dressing composition that can be used for effecting styling.

Hair care products for hair fixing that are most widely available on the cosmetics market include spray compositions in aerosols or pump-action bottles such as lacquers, sprays or mousses, for example, constituted of a solution that can be alcoholic or aqueous-alcoholic and of a film-forming polymer that can be water-soluble or alcohol-soluble, mixed with various cosmetic additives, or alternatively products that are to be applied by hand, such as gels, creams, pastes and waxes.

However, these hair styling formulations, for example, the aerosol sprays and lacquers, still may not give the hairstyle satisfactory resistance to the various natural everyday movements such as walking, movements of the head or gusts of wind. Moreover, these compositions may give the hair a sensation of stiffness, the so-called “helmet effect”. Styling products also include the styling shampoos, which can offer the benefit of combining washing of the hair while permitting shaping of the hair during drying, saving time for the increasingly busy user.

The polymers used conventionally for the formulation of hair styling products can be cationic, anionic, amphoteric or non-ionic film-forming polymers, which may lead to the formation of films that can be hard and brittle to a varying degree.

When the polymer is too brittle, the percentage elongation at break measured on the film may be low, e.g., generally less than 2%, and the hairstyle may not be long-lasting.

In an attempt to overcome this problem, these polymers have been mixed with plasticizers, providing films that can be more flexible and less brittle. These films can be quite deformable, and after deformation, they may only regain their initial form to a slight extent. Although the lasting properties of the hairstyle may be improved, they are still not satisfactory, since the form of the hairstyle changes, under the action of the stresses.

Therefore cosmetic compositions are being sought for the care and/or fixing of the hairstyle which can give the hair, in addition to real long-lasting fixing, good cosmetic properties, notably conditioning of the hair conferring at least one of good disentangling properties, softness and a pleasant, non-sticky appearance, ease of use and low risk in use.

### SUMMARY

Provided is a cosmetic composition comprising, at least one film-forming elastomeric, anionic polyurethane (A), wherein the at least one film-forming elastomeric, anionic polyurethane (A) comprises:

(i) at least one side chain comprising at least one ethylene oxide unit, wherein the at least one side chain
represents from 12 to 80% by weight of the polyurethane (A)
has a Mw ranging from 1000 g/mol to 30,000 g/mol

(ii) one main chain comprising at least one unit derived from:
a polypropylene glycol (PPG) and optionally a second non-ionic polyol;
a dihydroxy-carboxylic acid and
at least one di-isocyanates, and optionally
a chain extender organic polyamine having an average of at least two primary amine groups,

and at least one cationic fixing film forming polymer.

This composition provides a flexible, non-brittle film on human keratinous substances, which can follow their movements.

In addition the composition according to the disclosure provides conditioning of the hair.

These effects are long lasting effects.²

Also provided is a method of styling the hair comprising applying this composition to the hair.

### DETAILED DESCRIPTION

In the following text “at least one” is equivalent to the expression “one or several”

The Chemistry of Film-Forming Elastomeric Polyurethane

The film-forming elastomeric, anionic polyurethane (A) may be prepared according to the method described in PCT Patent Application Publications WO2006/124250 and WO03/087183.

In at least one embodiment, the film-forming elastomeric, anionic polyurethane (A) comprises

(i) at least one side chain comprising at least one ethylene oxide unit, wherein the at least one side chain

represents from 12 to 80% by weight of the polyurethane (A)

has a Mw ranging from 1000 g/mol to 30,000 g/mol

(ii) one main chain comprising at least one unit derived from:

a polypropylene glycol (PPG) and optionally a second non-ionic polyol;

a dihydroxy-carboxylic acid, such as dimethylolpropionic acid (DMPA);

at least one di-isocyanate, such as isophorone diisocyanate; and optionally

a chain extender organic polyamine having an average of at least two primary amine groups,

The polyurethane (A) is soluble or dispersible, either in an aqueous medium (100% water), or in a water/ethanol solvent mixture containing at most 30 wt. % of ethanol. For example, in some embodiments, at least 10 g of the polyurethane (A) is soluble or dispersible in 90 grams of water or of water/ethanol mixture.

The film-forming elastomeric, anionic polyurethane (A) can be partially or totally neutralized or over-neutralized with inorganic or organic bases, for example, with soda and/or triethylamine, in an amount making it possible to obtain, for example, a degree of neutralization of the anionic functions of the polymer from 0 to 150%, such as from 50 to 100%. The “degree of neutralization to 150%” is obtained with an amount of inorganic or organic base equal to 1.5 times the amount of base required to obtain the degree of neutralization to 100%.

In some embodiments, the film-forming elastomeric, anionic polyurethane (A) is provided in the form of a salt composed of two parts: one anionic polyurethane and a cationic ion, mineral or organic.

Mechanical Profile of the Film-Forming Elastomeric Polyurethane

In some embodiments, the film-forming elastomeric, anionic polyurethane (A) is chosen in such a way that the film obtained by drying of this polyurethane (A), at room temperature (24° C.±2° C.) and at a relative humidity of 48%±5%, has a mechanical profile defined by at least:

(a) an elongation at break (ε_{b}) greater than or equal to 730%±5% (e.g. −5% of 730% represents 694%), and/or

(b) an instantaneous recovery (Rᵢ) greater than or equal to 70%±5%, after elongation of 150%, and/or

(c) a recovery (R₃₀₀) at 300 seconds greater than or equal to 80%±5%.

### Measurement of the Parameters

For the purpose of the present disclosure, “film obtained by drying at room temperature (24° C.±2° C.) and at a relative humidity of 48%±5%” means the film obtained, in these conditions, from a mixture comprising 6% of active substance (a.s.) of film-forming elastomeric polyurethane (A), in a solvent (S). The amount of active substance is understood as being relative to the total weight of solvent (S) and the amount of the mixture is suitable for obtaining, in a Teflon matrix, a film with thickness of 500 μm±50 μm, after drying the mixture, with the drying being continued until there is no longer any change in weight of the film. The measurements are generally performed after at least 10 days of drying.

The solvent (S) is a mixture ethanol/water comprising less than 30% by weight of water.

For the purpose of the present disclosure, the “elongation at break” and the “degree of recovery” are evaluated by means of the tests described below.

To measure the instantaneous recovery and the recovery at 300 seconds, the polymer film obtained is cut into test pieces of rectangular shape, 80 mm long and 15 mm wide.

To measure the elongation, the polymer film obtained is cut into dumbbell-shaped test pieces.

The tests may be performed on apparatus marketed under the name Lloyd or marketed under the name Zwick in the same conditions of temperature and of humidity as for drying, e.g. a temperature of 24° C.±2° C. and a relative humidity of 48%±5%.

The test pieces are stretched at a speed of 20 mm/min and the distance between the jaws is 50 mm±1 mm.

The elongation at break represents the ratio of the maximum elongation of the film before it breaks to its initial length measured before undergoing deformation.

The procedure for determining the instantaneous recovery (Rᵢ) is as follows:

stretch the test piece 150% (εₘₐₓ) e.g. 1.5 times its initial length (I₀),

release the stress, imposing a return speed equal to the pulling speed, e.g. 20 mm/min, and measure the percentage elongation of the test piece, after returning to zero load (εᵢ).

The instantaneous recovery as a percentage (Rᵢ) is given by the following formula:

Rᵢ=((εₘₐₓ−εᵢ)/εₘₐₓ)×100

To determine the recovery at 300 seconds, it is held at zero stress for an additional 300 seconds, the test piece having undergone the preceding operations, and its percentage elongation is measured (ε₃₀₀ₛ).

The recovery at 300 seconds as a percentage (R₃₀₀ₛ) is given by the following formula:

R₃₀₀ₛ=((εₘₐₓ−ε₃₀₀ₛ)/εₘₐₓ)×100

In the compositions according to the disclosure, the at least one film-forming elastomeric, anionic polyurethane (A) is, for example, present at a concentration ranging from 0.01 to 20 wt. %, such as from 0.1 to 15 wt. %, and further such as from 1 to 10 wt. % relative to the total weight of the composition.

### Cationic Film Forming Fixing Polymers

The cationic film-forming fixing polymers for use according to the present disclosure are, for example, chosen from polymers having primary, secondary, tertiary and/or quaternary amine groups forming part of the polymer chain or joined directly to the latter, and having a molecular weight ranging from 500 to about 5,000,000, such as from 1000 to 3,000,000.

Among these polymers, non-limiting mention can be made of the following cationic polymers:

(1) the homopolymers or copolymers derived from acrylic or methacrylic esters or amides and having at least one of the units with the following formulae:

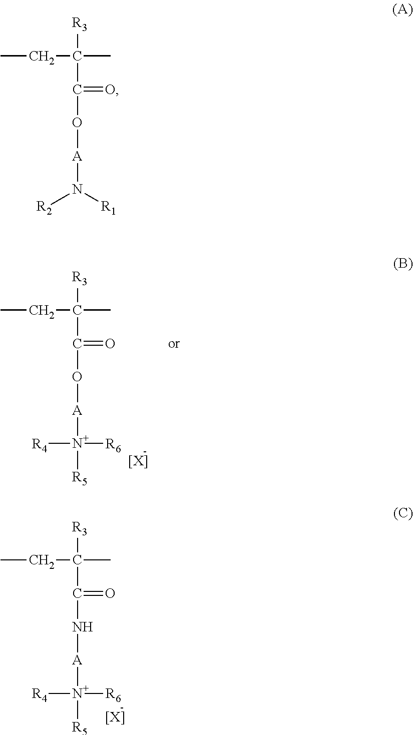

in which:

R₃ denotes a hydrogen atom or a CH₃ radical;

A is a linear or branched alkyl group having from 1 to 6 carbon atoms or a hydroxyalkyl group having from 1 to 4 carbon atoms;

R₄, R₅, R₆, which may be identical or different, represent an alkyl group having from 1 to 18 carbon atoms or a benzyl radical;

R₁ and R₂, which may be identical or different, each represent a hydrogen atom or an alkyl group having from 1 to 6 carbon atoms; and

X denotes a methosulphate anion or a halide such as chloride or bromide.

The copolymers of family (1) additionally contain at least one unit derived from comonomers, which can be chosen from the family comprising the acrylamides, methacrylamides, diacetone-acrylamides, acrylamides and methacrylamides substituted on the nitrogen with lower alkyl groups (C1-C4), groups derived from acrylic or methacrylic acids or their esters, vinyl lactams such as vinyl pyrrolidone or vinyl caprolactam, vinyl esters.

Thus, among these copolymers of family (1), further non-limiting examples include:

the copolymers of acrylamide and dimethylaminoethyl methacrylate quaternized with dimethyl sulphate or with a dimethyl halide, such as that sold under the name HERCOFLOC® by the company HERCULES,

the copolymers of acrylamide and methacryloyloxyethyltrimethylammonium chloride described for example in patent application EP-A-080976 and sold under the name BINA QUAT P 100 by the company CIBA GEIGY,

the copolymers of acrylamide and methacryloyloxyethyltrimethylammonium methosulphate such as that sold under the name RETEN by the company HERCULES,

the copolymers of esters of acrylic or methacrylic acid with dialkylaminoalkyl methacrylate or dialkylaminoalkyl acrylate eventually with others monomers and eventually crosslinked such CARBOPOL AQUA CC POLYMER from LUBRIZOL.

the vinyl pyrrolidone/dialkylaminoalkyl acrylate or methacrylate copolymers, quaternized or unquaternized, such as the products sold under the name “GAFQUAT®” by the company ISP for example “GAFQUAT® 734” or “GAFQUAT® 755” or else the products called “COPOLYMER® 845, 958 and 937”. These polymers may be described in detail in French Patent Nos. 2 077 143 and 2 393 573,

the fatty-chain polymers with a vinyl pyrrolidone unit, such as the products sold under the name Styleze W20 and Styleze W10 by the company ISP,

the dimethylaminoethyl methacrylate/vinyl caprolactam/vinyl pyrrolidone terpolymers such as the product marketed under the name GAFFIX VC 713 by the company ISP, and

the quaternized vinyl pyrrolidone/dimethylaminopropyl methacrylamide copolymers such as the products marketed under the name “GAFQUAT® HS100” by the company ISP;

(2) the non-cellulosic cationic polysaccharides, for example, with quaternary ammonium such as those described in U.S. Pat. Nos. 3,589,578 and 4,031,307 such as the guar gums containing cationic trialkylammonium groups. Such products are marketed, for example, under the trade names JAGUAR C13 S, JAGUAR C 15, JAGUAR C 17 by the company MEYHALL.

(3) the quaternary copolymers of vinyl pyrrolidone and vinyl imidazole;

(4) the chitosans or their salts; the salts that can be used, for example, are the acetate, lactate, glutamate, gluconate or pyrrolidone-carboxylate of chitosan.

Among these compounds, further non-limiting mention can be made of chitosan having a degree of deacetylation of 90.5 wt. % sold under the name KYTAN BRUT STANDARD by the company ABER TECHNOLOGIES, the chitosan pyrrolidone-carboxylate marketed under the name KYTAMER® PC by the company AMERCHOL.

(5) the cationic derivatives of cellulose such as the copolymers of cellulose or of cellulose derivatives grafted with a water-soluble monomer bearing a quaternary ammonium, and described, for example, in U.S. Pat. No. 4,131,576, for instance the hydroxyalkyl celluloses, such as the hydroxymethyl, hydroxyethyl or hydroxypropyl celluloses grafted notably with a methacryloyloxyethyl trimethylammonium salt, methacrylamidopropyl trimethylammonium salt, or dimethyl-diallylammonium salt.

The commercial products corresponding to this definition are, for example, the products sold under the name “CELQUAT L 200” and “CELQUAT H 100” by the company National Starch.
Other polymers of this class are cellulosic cationic polymer containing hydrophobic groups such as alkyl groups with 8 to 30 carbon atoms.
Commercial products of this type are sold under denominations QUATRISOFT LM 200 and SOFCAT SL 100 by HERCULES.

Cationic polymers are more preferably chosen among types (1) or (5).

The cationic polymers preferably represent from 0.01 to 10 in weight, better 0.1 to 5% wt relative to the total weight of the composition.

### Acid Derivative

Advantageously, the composition further comprises at least one α-hydroxy or α-keto acid.

### The α-Hydroxy Acids are Preferably Chosen Among Compounds of General Formula (I):

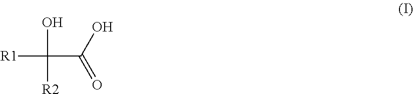

Formulation (I) in which
R1 represents one atom or one group chosen among H, OH, NH2, CH2-COOH or a linear or branched C1-C4 alkyl,
R2 represents one atom or one group chosen among H, COOH, CHOH—COOH, CF3, CH═CH2, NHCONH2, a linear, branched or cyclic C₁-C₈ alkyl optionally substituted with a radical chosen from OH, Cl, NH2, COOH, CF3 and SCH3;
a phenyl or benzyl optionally substituted with one OH or OCH3 radical;
or alternatively the radical

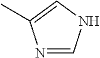

R1 and R2 may also together form an oxo radical (═O) or a cyclopropyl, cyclobutyl, hydroxycyclobutyl, cyclopentyl or cyclohexyl ring with the carbon atom that bears them, or alternatively a radical

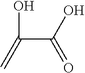

when R1 designs H, R2 may also represent a (CHOH)2CH2OH or (CHOH)3CH2OH radical, and the stereoisomers, organic or mineral salts and solvates thereof.

### Preferred Compounds of Formula (I) are:

glycolic acid
oxalic acid
lactic acid
1-hydroxy-1-cyclopropanecarboxylic acid
2-hydroxy-3-butenoic acid
2-hydroxyisobutyric acid
2-hydroxy-n-butyric acid
isoserine
glyceric acid
2-hydroxy-3-methylbutyric acid
2-hydroxy-2-methylbutyric acid
2-hydroxyvaleric acid
4-amino-2-hydroxybutyric acid
1-hydroxycyclohexanecarboxylic acid
dihydroxyfumaric acid
citramalic acid
tartaric acid
citric acid
2-hydroxy-4-(methylthio)butyric acid
mandelic acid
2-hydroxy-3-methylvaleric acid
glyoxylurea
β-imidazolelactic acid
2-trifluoromethyl-2-hydroxypropionic acid
hexahydromandelic acid
2-hydroxyoctanoic acid
arabic acid
3-phenylactic acid hydroxyphenylglycine
3-hydroxymandelic acid
4-hydroxymandelic acid
2-hydroxynonanoic acid
L-arginic acid
3-methoxymandelic acid
4-methoxymandelic acid
3-(4-hydroxyphenyl)lactic acid
tartronic acid
tartaric acid
β-chlorolactic acid
1-cylcopentanol-1-carboxylic acid
1,2-dihydroxycyclobutanecarboxylic acid
2-ethyl-2-hydroxybutric acid
α-hydroxyisocaproic acid
α-hydroxycaproic acid
2-hydroxy-3,3-dimethylbutyric acid
malic acid
hydroxytartronic acid
gluconic acid
   and the stereoisomers, and solvates thereof.

### More Particularly Preferred Compounds of Formula (I) are :

glycolic acid
oxalic acid
lactic acid
malic acid
citric acid
tartaric acid
glyceric acid
arabic acid
gluconic acid
hydroxytartronic acid

### α-Keto are Preferably Chosen Among Compounds of General Formula (II):

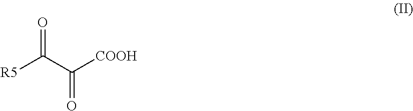

R5 represents COOH group, a linear or branched C1-C6 alkyl group optionally substituted with an OH, COOH or Br radical; a phenyl or benzyl group optionally substituted with an OH or COOH radical; or an indolyl radical
and the stereoisomers, and solvates thereof.

### Preferred Compounds of Formula (II) are:

3-indoleglyoxalic acid
imidazolopyruvice acid HCl
2-keto-L-gulonic acid
2-carboxy-α-oxobenzeneacetic acid
3-indolepyruvic acid
2-ketoglutaric acid dihydrate
   and the stereoisomers, organic or mineral salts and solvates thereof.

### More Particularly Preferred Compounds of Formula (II) are:

pyruvic acid
2-ketobutyric acid
β-hydroxypyruvic acid
ketomalonic acid
oxolacetic acid
2-ketoglutaric acid
2-keto-L-gulonic acid
2-ketoglutaric acid dihydrate
   Preferably α-hydroxy or α-ketoacids are chosen among α-hydroxy acids and more particularly among lactic acid, malic acid, citric acid, gluconic acid and tartaric acid.
   These α-hydroxy or α-keto-acids preferably represent from 0.1 to 10, better from 0.2 to 5% wt relative to the total weight of the composition.
   pH
   The working pH is preferably less than or equal to 9 and more particularly less than or equal to 7. It is adjusted by acidic compounds such as α-hydroxy or α-keto acids previously cited and by the help of other acidic compounds, such as organic acids (acetic acid by example), mineral acids or alcalin compounds such as alcanolamines, ammonia, metal, carbonates.

### Surfactants

The composition may further contain at least one surfactant. The at least one surfactant for use in the composition according to the present disclosure can be chosen from anionic, non-ionic, amphoteric and cationic surfactants.

Among the anionic surfactants that can be used, individually or mixed, within the scope of the present disclosure, non-limiting mention can be made of the following compounds: alkyl sulphates, alkyl ether sulphates, alkylamidoether sulphates, alkarylpolyether sulphates, monoglyceride-sulphates; alkyl sulphonates, alkylamide sulphonates, alkaryl sulphonates, α-olefin-sulphonates, paraffin-sulphonates; alkyl sulphosuccinates, alkyl ether sulphosuccinates, alkylamide-sulphosuccinates; alkyl sulphoacetates; acyl sarconisates; and the acylglutamates, the alkyl and acyl groups of all these compounds having from 6 to 24 carbon atoms and the aryl group, such as those denoting a phenyl or benzyl group, these compounds comprising a counterion preferably selected from ammonium ion, ions of amines such as aminoalcohols, alkalimetal ions such as sodium, alkaline earl metal such as magnesium.

Exemplary mention may also be made of, within the scope of the present disclosure, C₆-C₂₄ alkyl esters of polyglycoside-carboxylic acids such as alkyl glucoside-citrates, alkyl polyglycoside-tartrates, and alkyl polyglycoside-sulphosuccinates; alkylsulphosuccinamates, acylisethionates and N-acyltaurates, the alkyl or acyl group of all these compounds having from 12 to 20 carbon atoms. Among other anionic surfactants that can be used, further nonlimiting mention can be made of the acyl lactylates in which the acyl group has from 8 to 20 carbon atoms.

Moreover, the anionic surfactants include but are not limited to the alkyl-D-galactoside uronic acids and their salts as well as the polyoxyalkylenated alkyl(C₆-C₂₄)ether-carboxylic acids, the polyoxyalkylenated alkyl(C₆-C₂₄)aryl(C₆-C₂₄)ether-carboxylic acids, the polyoxyalkylenated alkyl(C₆-C₂₄)amidoether carboxylic acids and their salts, for example, those having from 2 to 50 ethylene oxide groups, and mixtures thereof.

The non-ionic surfactants that can be used within the scope of the present disclosure may be compounds known in the art (in this connection, see, for example, “Handbook of Surfactants” by M. R. PORTER, publ. Blackie & Son (Glasgow and London), 1991, pp 116-178). They, for example, can be chosen from the alcohols, the alpha-diols, the alkyl(C₁-C₂₀) phenols, these compounds being polyethoxylated, polypropoxylated or polyglycerolated and having at least one fatty chain with for example from 8 to 18 carbon atoms, with the number of ethylene oxide groups or propylene oxide groups notably ranging from 2 to 50 and the number of glycerol groups for example, ranging from 2 to 30. Non-limiting mention can also be made of the copolymers of ethylene oxide and propylene oxide, the condensates of ethylene oxide and propylene oxide on fatty alcohols; the polyethoxylated fatty amides, for example, having from 2 to 30 mol of ethylene oxide; the polyglycerolated fatty amides having on average 1 to 5 glycerol groups, such as 1.5 to 4; the ethoxylated sorbitan fatty acid esters having from 2 to 30 mol of ethylene oxide; the sucrose fatty acid esters, the polyethylene glycol fatty acid esters, ethoxylated vegetal oils; the alkyl(C₆-C₂₄)polyglucosides, the derivatives of N-alkyl(C₆-C₂₄)glucamine, the oxides of amines such as the oxides of alkyl(C₁₀-C₁₄)amines or the oxides of N-acyl(C₁₀-C₁₄)aminopropylmorpholine; and mixtures thereof.

The amphoteric surfactants, suitable for the present disclosure, can, for example, be derivatives of secondary or tertiary aliphatic amines eventually quaternized, in which the aliphatic group is a linear or branched chain having 8 to 22 carbon atoms and containing at least one water-solubilizing anionic group such as, for example, a carboxylate, sulphonate, sulphate, phosphate or phosphonate group. Non-limiting mention can also be made of the alkyl(C₈-C₂₀)betaines, the sulphobetaines, the alkyl(C₈-C₂₀)amidoalkyl(C₆-C₈)-betaines or the alkyl(C₈-C₂₀)amidoalkyl(C₆-C₈)sulphobetaines; and mixtures thereof.

Among the derivatives of amines, non-limiting examples include the products marketed under the name MIRANOL®, as described in U.S. Pat. Nos. 2,528,378 and 2,781,354 and classified in the CTFA Dictionary, 3rd edition, 1982, under the designations Amphocarboxy-glycinate and Amphocarboxypropionate with the respective structures (1) and (2):

R₂—CONHCH₂CH₂—N⁺(R₃)(R₄)(CH₂COO⁻) (1)

in which:

R₂ represents an alkyl or alkenyl C10-C30 group derived from an R₂—COOH acid , preferably a group present in hydrolysed copra oil or a group heptyl, nonyl or undecyl group,

R₃ represents a beta-hydroxyethyl group, and

R₄ represents a carboxymethyl group;

and

R₂—CONHCH₂CH₂—N(B)(C) (2)

in which:

B represents —CH₂CH₂OX′,

C represents —(CH₂)_{z}—Y′, with z=1 or 2,

X′ represents the group —CH₂CH₂—COOH—CH₂COOZ′, CH₂CH₂—COOH, —CH₂CH₂—COOZ′, or a hydrogen atom,

Y′ represents —COOH or the group —CH₂—CHOH—SO₃H,

Z′ represents ion issued from a metal such as sodium, an ammonium group, or ion issued from an amine

R₂ represents the alkyl or alkenyl group of an R_{2′}—COOH acid, preferably a group present in copra oil or in hydrolysed linseed oil, an alkyl group, for example, in C₁₇ and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

As an example, the cocoamphodiacetate is marketed under the trade name MIRANOL® “C2M concentré” by the company RHODIA.

Among the amphoteric surfactants, the alkyl(C₈-C₂₀)betaines such as cocobetaine, the alkyl(C₈-C₂₀)amidoalkyl(C₈-C₈)betaines such as cocamidobetaine, the alkylamphodiacetates such as disodium cocoamphodiacetate, and mixtures thereof, can be exemplarily used.

The composition according to the disclosure can additionally contain at least one cationic surfactant, such as the salts of primary, secondary or tertiary fatty amines, optionally polyoxyalkylenated, the quaternary ammonium salts such as the tetraalkylammonium, alkylamidoalkyltrialkyl-ammonium, trialkylbenzylammonium, trialkylhydroxyalkyl-ammonium or alkylpyridinium chlorides or bromides, the derivatives of imidazoline ,quaternary ammonium salts with at least one ester function, all of these above mentioned compounds containing a least one C8C30 fatty chain, saturated or unsaturated.

Among these cationic surfactants the following compounds are preferred: salts of behenyl trimethylammonium or of cetyltrimethyl ammonium and salts of methyl ammonium.

The non-ionic, anionic, amphoteric and cationic surfactants described above can be used individually or mixed and their individual amount ranges from 0.01 to 30 wt. %, such as from 0.05 to 20 wt. % and further such as from 0.1 to 10 wt. % relative to the total weight of the composition.

### Silicones

In some embodiments, the composition may comprise at least one silicone. The silicones for use as additives in the cosmetic compositions of the present disclosure are, for example, volatile or non-volatile silicones, cyclic, linear or branched, unmodified or modified with organic groups

The silicones that can be used according to the disclosure can be soluble or insoluble in the composition and, for example, can be polyorganosiloxanes that may be insoluble in the composition of the disclosure. They can be in the form of oils, waxes, resins or gums.

The organopolysiloxanes are described in more detail in the work of Walter NOLL “Chemistry and Technology of Silicones” (1968), Academic Press. They can be volatile or non-volatile.

When they are volatile, the silicones are, for example, chosen from those with a boiling point from 60° C. to 260° C., and further for example, from:

(i) the cyclic silicones having from 3 to 7, for example, 4 or 5 silicon atoms. These are, for example, the octamethylcyclotetrasiloxane marketed, for example, under the name VOLATILE SILICONE® 7207 by UNION CARBIDE or SILBIONE® 70045 V2 by RHODIA, the decamethylcyclopentasiloxane marketed under the name VOLATILE SILICONE® 7158 by UNION CARBIDE, and SILBONE® 70045 V5 by RHODIA, and mixtures thereof.

Non-limiting mention can also be made of the cyclocopolymers of the dimethylsiloxane/methylalkylsiloxane type, such as the SILICONE VOLATILE® FZ 3109 marketed by the company UNION CARBIDE, of formula:

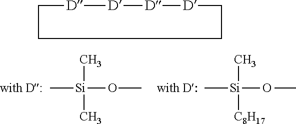

Further non-limiting mention can also be made of the mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and oxy-1,1′-(hexa-2,2,2′2′3,3′-trimethylsilyloxy)bis-neopentane;

(ii) the volatile linear silicones having 2 to 9 silicon atoms and with a viscosity less than or equal to 5.10⁻⁶ m²/s at 25° C. These include, for example, the decamethyltetrasiloxane marketed, for example, under the name “SH 200” by the company TORAY SILICONE. Silicones included in this class may also be described in the article published in Cosmetics and Toiletries, Vol. 91, Jan. 76, p. 27-32—TODD & BYERS “Volatile Silicone fluids for cosmetics”.

Non-volatile silicones are exemplarily used in some embodiments, for example, polyalkylsiloxanes, polyarylsiloxanes, polyalkarylsiloxanes, silicone gums and resins, polyorganosiloxanes modified with organofunctional groups and mixtures thereof.

These silicones are, for example, chosen from the polyalkylsiloxanes, among which non-limiting mention can be made of the polydimethylsiloxanes with trimethylsilyl end groups. The viscosity of the silicones is measured at 25° C. according to standard ASTM 445 Appendix C.

Among these polyalkylsiloxanes, further non-limiting mention can be made of, the following commercial products:

the SILBIONE® oils of series 47 and 70 047 or the MIRASIL® oils marketed by RHODIA, for example the oil 70 047 V 500 000;

the oils of the MIRASIL® series marketed by the company RHODIA;

the oils of the 200 series from the company DOW CORNING such as DC200 with a viscosity of 60 000 mm²/s;

the VISCASIL® oils from GENERAL ELECTRIC and certain oils of the SF series (SF 96, SF 18) from GENERAL ELECTRIC.

Even further non-limiting mention can be made of the polymethylsiloxanes with dimethylsilanol end groups known under the name dimethiconol (CTFA), such as the oils of series 48 from the company RHODIA.

In this class of polyalkylsiloxanes, non-limiting mention can be made of the products marketed under the names “ABIL WAX® 9800 and 9801” by the company GOLDSCHMIDT, which are polyalkyl(C₁-C₂₀)siloxanes.

The polyalkarylsiloxanes are chosen, for example, from the polydimethyl/methylphenylsiloxanes, the polydimethyl/diphenylsiloxanes, linear and/or branched with viscosity ranging from 1.10⁻⁵ to 5.10⁻² m²/s at 25° C.

Among these polyalkarylsiloxanes, non-limiting mention can be made of the products marketed under the following names:

the SILBIONE® oils of the 70 641 series from RHODIA;

the oils of the series RHODORSIL® 70 633 and 763 from RHODIA;

the oil DOW CORNING 556 COSMETIC GRADE FLUID from DOW CORNING;

the silicones of the PK series from BAYER such as the product PK20;

the silicones of the PN, PH series from BAYER such as the products PN1000 and PH1000; and

certain oils of the SF series from GENERAL ELECTRIC such as SF 1023, SF 1154, SF 1250, SF 1265.

The silicone gums that can be used according to the disclosure are, for example, polyorganosiloxanes, with high number-average molecular weights ranging from 200 000 to 1 000 000 used alone or mixed in a solvent. In some embodiments, the solvent is chosen from the volatile silicones, the polydimethylsiloxane (PDMS) oils, the polyphenylmethylsiloxane (PPMS) oils, the isoparaffins, the polyisobutylenes, methylene chloride, pentane, dodecane, tridecane and mixtures thereof.

Further non-limiting mention can be made of the following products:

polydimethylsiloxane gums,

polydimethylsiloxane/methylvinylsiloxane gums,

polydimethylsiloxane/diphenylsiloxane gums,

polydimethylsiloxane/phenylmethylsiloxane gums,

polydimethylsiloxane/diphenylsiloxane/methylvinylsiloxane gums.

Products that can be used as non-limiting examples according to the disclosure are mixtures such as:

mixtures formed from a polydimethylsiloxane hydroxylated at the chain end, or dimethiconol (CTFA) and a cyclic polydimethylsiloxane also called cyclomethicone (CTFA) such as the product Q2 1401 marketed by the company DOW CORNING;

mixtures of a polydimethylsiloxane gum and a cyclic silicone such as the product SF 1214 Silicone Fluid from the company GENERAL ELECTRIC; which is a gum SF 30 corresponding to a dimethicone, having a number-average molecular weight of 500 000 dissolved in oil SF 1202 Silicone Fluid corresponding to decamethylcyclopentasiloxane;

mixtures of two PDMS with different viscosities, such as a mixture of a PDMS gum and a PDMS oil, such as the product SF 1236 from the company GENERAL ELECTRIC. The product SF 1236 is a mixture of a gum SE 30 described above having a viscosity of 20 m²/s and an oil SF 96 with a viscosity of 5.10⁻⁶ m²/s. This product, for example, has 15% of gum SE 30 and 85% of oil SF 96.

The resins of organopolysiloxanes that can be used according to the disclosure include but are not limited to crosslinked siloxane systems containing the units:

R₂SiO_{2/2}, R₃SiO_{1/2}, RSiO_{3/2} and SiO_{4/2}

in which R represents a hydrocarbon group with 1 to 16 carbon atoms or a phenyl group. Among these products, exemplary mention can be made of the ones in which R denotes a C₁-C₄ lower alkyl group, such as methyl or a phenyl group.

Among these resins, non-limiting mention can be made of the product marketed under the name “DOW CORNING 593” or those marketed under the names “SILICONE FLUID SS 4230 and SS 4267” by the company GENERAL ELECTRIC and which are silicones of dimethyl/trimethyl siloxane structure.

Further non-limiting mention can be made of the resins of the trimethylsiloxysilicate type marketed notably under the names X22-4914, X21-5034 and X21-5037 by the company SHIN-ETSU.

The organically modified silicones that can be used as non-limiting examples according to the disclosure are silicones as discussed previously, having in their structure at least one organofunctional group fixed by means of a hydrocarbon group.

Among the organically modified silicones, non-limiting mention can be made of polyorganosiloxanes having:

polyethyleneoxy and/or polypropyleneoxy groups optionally with C₆-C₂₄ alkyl groups such as the products called dimethicone copolyol marketed by the company DOW CORNING under the name DC 1248 or the SILWET® oils L 722, L 7500, L 77, L 711 from the company UNION CARBIDE and the alkyl (C₁₂)-methicone copolyol marketed by the company DOW CORNING under the name Q2 5200;

substituted or unsubstituted amine groups such as the products marketed under the name GP 4 Silicone Fluid and GP 7100 by the company GENESEE or the products marketed under the names Q2 8220 and DOW CORNING 929 or 939, or DOW CORNING 2-8299 by the company DOW CORNING or the product marketed under the name BELSIL ADM LOG 1 by the company WACKER. The substituted amine groups are, for example, C₁-C₄ aminoalkyl groups;

thiol groups, such as the products marketed under the names GP 72A and GP 71 from GENESEE.

alkoxylated groups, such as the product marketed under the name “SILICONE COPOLYMER F-755” by SWS SILICONES and ABIL WAX® 2428, 2434 and 2440 by the company GOLDSCHMIDT;

hydroxylated groups, such as the polyorganosiloxanes with hydroxyalkyl function described in French Patent Application No. FR 8 516 334 A;

alkoxyalkyl groups, for example the polyorganosiloxanes described in U.S. Pat. No. 4,957,732 A;

anionic groups of the carboxyl type, for example as in the products described in European Patent No. EP 186 507 of the company CHISSO CORPORATION, or of alkyl-carboxyl type such as those present in the product X-22-3701E from the company SHIN-ETSU; 2-hydroxyalkylsulphonate; 2-hydroxyalkylthiosulphate such as the products marketed by the company GOLDSCHMIDT under the names ABIL® S201 and ABIL® S255;

hydroxyacrylamino groups, such as the polyorganosiloxanes described in European Patent Application No. EP 342 834. For example, the product Q2-8413 from the company DOW CORNING.

halogenated silicones.

The silicones as described above can be used alone or mixed, in an individual amount from 0.01 to 20 wt. %, such as from 0.1 to 5 wt. %.

### Non-Silicone Fatty Substances

In some embodiments, the compositions of the disclosure may also contain at least one non-silicone fatty substance, which can be chosen from mineral, vegetable, animal and synthetic oils; waxes, fatty esters, ethoxylated and non-ethoxylated fatty alcohols, and fatty acids.

As oils for use in the composition of the disclosure, non-limiting examples include:

hydrocarbon oils of animal origin, such as perhydrosqualene;

hydrocarbon oils of vegetable origin, such as the liquid triglycerides of fatty acids having from 4 to 10 carbon atoms such as the triglycerides of heptanoic or octanoic acids or alternatively, for example, sunflower oil, maize oil, soya oil, cucurbit oil, grapeseed oil, sesame oil, hazelnut oil, apricot oil, macadamia oil, arara oil, sunflower oil, castor oil, avocado oil, the triglycerides of caprylic/capric acids such as those sold by the company Stearineries Dubois or those sold under the names Miglyol® 810, 812 and 818 by the company Dynamit Nobel, jojoba oil, shea butter oil;

linear or branched hydrocarbons, of mineral or synthetic origin, such as the volatile or non-volatile paraffin oils, and their derivatives, petroleum jelly, the polydecenes, hydrogenated polyisobutene such as Parleam®; the isoparaffins such as isohexadecane and isodecane.

the fluorinated oils, partially hydrocarbonized and/or siliconized, such as those described in document JP-A-2-295912; as fluorinated oils, non-limiting example include perfluoromethylcyclopentane and perfluoro-1,3-dimethylcyclohexane, sold under the names “FLUTEC® PC1” and “FLUTEC® PC3” by the company BNFL Fluorochemicals; perfluoro-1,2-dimethylcyclobutane; perfluoroalkanes such as dodecafluoropentane and tetradecafluorohexane, sold under the names “PF 5050®” and “PF 5060®” by the company 3M, or alternatively bromoperfluorooctyl sold under the name “FORALKYL®” by the company Atochem; nonafluoromethoxybutane and nonafluoroethoxyisobutane; derivatives of perfluoromorpholine, such as 4-trifluoromethyl perfluoromorpholine sold under the name “PF 5052®” by the company 3M;

The wax or waxes are, for example, chosen from carnauba wax, candelilla wax, and Alpha wax, paraffin wax, ozokerite, the vegetable waxes such as olive wax, rice wax, hydrogenated jojoba wax and the absolute waxes of flowers such as the essential wax of blackcurrant flower sold by the company BERTIN (France), animal waxes such as beeswax, and modified beeswax (cerabellina); other waxes and waxy raw materials that can be used according to the disclosure are, for example the marine waxes such as that sold by the company SOPHIM under reference M82, the waxes of polyethylene or of polyolefins in general.

The saturated or unsaturated fatty acids are differents from α-hydroxy or α-cetoacids above mentioned and are , for example, chosen from myristic acid, palmitic acid, stearic acid, behenic acid, oleic acid, linoleic acid, linolenic acid and isostearic acid.

The fatty esters are, for example, the esters of carboxylic acids, such as the mono-, di-, tri- or tetracarboxylates.

The esters of carboxylic acids are, for example, the esters of saturated or unsaturated, linear or branched C₁-C₂₆ aliphatic acids and of saturated or unsaturated, linear or branched C₁-C₂₆ aliphatic alcohols, the total number of carbons in the esters being greater than or equal to 10.

Among the monoesters, non-limiting mention can be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, ethyl-2-hexyl palmitate, 2-octyldecyl palmitate, alkyl myristates such as isopropyl, butyl, cetyl, 2-octyldodecyl myristate, hexyl stearate, butyl stearate, isobutyl stearate; dioctyl malate, hexyl laurate, and 2-hexyldecyl laurate.

Non-limiting examples also include the esters of C₄-C₂₂ di- or tricarboxylic acids and of C₁-C₂₂ alcohols and the esters of mono-, di- or tricarboxylic acids and of C₂-C₂₆ di-, tri-, tetra- or pentahydroxy alcohols.

Further non-limiting mention can be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisotearyl citrate; glyceryl trilactate; glyceryl trioctanoate; trioctyldodecyl citrate; trioleyl citrate; propylene glycol dioctanoate and neopentyl glycol diheptanoate; the esters mentioned above being different from the esters of formula (I).

Among the esters mentioned above, even further non-limiting mention can be made of the ethyl and isopropyl palmitates, ethyl-2-hexyl palmitate, 2-octyldecyl palmitate, alkyl myristates such as isopropyl, butyl, cetyl, 2-octyldodecyl myristate, hexyl stearate, butyl stearate, isobutyl stearate; dioctyl malate, hexyl laurate, 2-hexyldecyl hexyl and isononyl isononanate, and cetyl octanoate.

For fatty alcohols, non-limiting mention can be made of saturated or unsaturated, linear or branched fatty alcohols having from 8 to 26 carbon atoms, such as cetyl alcohol, stearyl alcohol and their mixture (cetylstearyl alcohol), octyldodecanol, 2-butyloctanol, 2-hexyldecanol, 2-undecylpentadecanol, oleic alcohol or linoleic alcohol.

The fatty substances generally individually can represent from 0.1 to 50%; such as from 1 to 30%, and further such as from 2 to 20 wt. % of the total composition.

### Further Fixing Film Forming Polymer

The compositions can further include at least one additional anionic, amphoteric and non-ionic fixing polymer different from the film-forming elastomeric polyurethane (A) of the disclosure. Fixing polymer means, for the purpose of the present disclosure, any polymer for imparting shape to the hair or for maintaining the hair in a given shape.

The fixing polymers can be soluble in the cosmetically acceptable medium or insoluble in the medium and used in this case in the form of dispersions of solid or liquid particles of polymer (latex or pseudolatex).

The anionic fixing polymers generally used are polymers having groups derived from carboxylic, sulphonic or phosphoric acid and have a number-average molecular weight from about 500 to 5,000,000.

The carboxyl groups are supplied by unsaturated mono- or dicarboxylic acid monomers such as those corresponding to the formula:

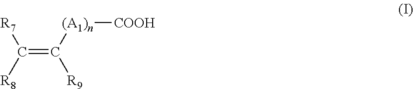

in which n is an integer from 0 to 10, A₁ denotes a methylene group, optionally joined to the carbon atom of the unsaturated group or to the adjacent methylene group when n is greater than 1, via a heteroatom such as oxygen or sulphur, R₇ denotes a hydrogen atom, a phenyl or benzyl group, R₈ denotes a hydrogen atom, a lower alkyl group or carboxyl, R₉ denotes a hydrogen atom, a lower alkyl group, a —CH₂—COOH, phenyl or benzyl group.

In the aforementioned formula, a lower alkyl group, for example, denotes a C₁-C₄ alkyl, such as the methyl and ethyl groups.

The anionic fixing polymers with carboxyl groups according to the disclosure include but are not limited to:

A) The copolymers of acrylic acid and of acrylamide sold in the form of their sodium salts under the names RETEN 421, 423 or 425 by the company HERCULES, the sodium salts of the polyhydroxycarboxylic acids;

B) The copolymers of acrylic or methacrylic acid with a monoethylenic monomer such as ethylene, styrene, vinyl esters, esters of acrylic or methacrylic acid, optionally grafted on a polyalkylene glycol such as polyethylene glycol, and optionally crosslinked. Such polymers may be described, for example, in French patent No. 1 222 944 and German Patent Application No. 2 330 956, the copolymers of this type having in their chain an acrylamide unit optionally N-alkylated and/or hydroxyalkylated, as described, for example, in Luxembourg Patent Applications Nos. 75370 and 75371 or offered under the name QUADRAMER by the company AMERICAN CYANAMID. Non-limiting mention can be made of the acrylic acid/ethyl acrylate/N-tert-butylacrylamide terpolymers such as ULTRAHOLD STRONG sold by the company BASF. Non-limiting examples also include the copolymers of acrylic acid and of C₁-C₄ alkyl methacrylate and the terpolymers of vinyl pyrrolidone, acrylic acid and C₁-C₂₀ alkyl methacrylate, for example lauryl methacrylate, such as that marketed by the company ISP under the name ACRYLIDONE® LM and the methacrylic acid/ethyl acrylate/tert-butyl acrylate terpolymers such as the product marketed under the name LUVIMER® 100 P by the company BASF.

Further non-limiting mention can be made of the methacrylic acid/acrylic acid/ethyl acrylate/methyl methacrylate copolymers in aqueous dispersion, marketed under the name AMERHOLD® DR 25 by the company AMERCHOL;

C) The copolymers of crotonic acid, such as those having vinyl acetate or propionate units in their chain, and optionally other monomers such as the allyl or methallyl esters, vinyl ether or vinyl ester of a saturated, linear or branched carboxylic acid, with long hydrocarbon chain, such as those having at least 5 carbon atoms, and the polymers can optionally be grafted or crosslinked, or alternatively another vinyl, allyl or methallyl ester monomer of an α- or β-cyclic carboxylic acid. Such polymers may be described in, among others, French Patent Nos. 1 222 944, 1 580 545, 2 265 782, 2 265 781, 1 564 110 and 2 439 798. Commercial products included in this class can be the resins 28-29-30, 26-13-14 and 28-13-10 marketed by the company National Starch;

D) The copolymers of C₄-C₈ monounsaturated carboxylic acids or anhydrides chosen from:
copolymers comprising (i) at least one maleic, fumaric, itaconic acid and/or anhydrides and (ii) at least one monomer chosen from vinyl esters, vinyl ethers, vinyl halides, phenylvinylic derivatives, acrylic acid and its esters, anhydride functions of these copolymers being optionally monoesterified or monoamidated. Such polymers may be described, for example, in U.S. Pat. Nos. 2,047,398, 2,723,248, and 2,102,113, GB Patent No. 839 805. Commercial products are, for example, those sold under the names GANTREZ® AN or ES by the company ISP;
copolymers comprising (i) at least one maleic, citraconic, itaconic acid and/or anhydride unit and (ii) at least one monomer chosen from allyl and methallyl esters optionally with at least one acrylamide, methacrylamide, α-olefin groups, acrylic or methacrylic esters, acrylic or methacrylic acids or vinyl pyrrolidone in their chain,

the anhydride functions of these copolymers being optionally monoesterified or monoamidated.

These polymers may be for example described in French Patent Nos. 2 350 384 and 2 357 241 of the applicant;

E) Polyacrylamides having carboxylate groups.

F) Homopolymers and copolymers containing sulphonic groups such as the polymers having vinylsulphonic, styrene-sulphonic, naphthalene-sulphonic or acrylamido-alkylsulphonic units different from the branched sulphonic polyesters of the invention.

These polymers can, for example, be chosen from:

salts of polyvinylsulphonic acid having a molecular weight ranging from about 1000 to 100,000, as well as copolymers with an unsaturated comonomer such as acrylic or methacrylic acids and their esters, as well as acrylamide or its derivatives, vinyl ethers and vinyl pyrrolidone;

salts of polystyrene-sulphonic acid such as the sodium salts sold for example under the name Flexan® 500 and Flexan® 130 by National Starch. These compounds my be described in French Patent No. FR 2 198 719;

salts of polyacrylamide-sulphonic acids such as those mentioned in U.S. Pat. No. 4,128,631, and for example, the polyacrylamidoethylpropane-sulphonic acid sold under the name COSMEDIA POLYMER HSP 1180 by HENKEL.

For another anionic fixing polymer that can be used according to the disclosure, non-limiting examples include the branched anionic block polymer sold under the name Fixate™ G-100 Polymer by the company Lubrizol.

According to the disclosure, the anionic fixing polymers are for example, chosen from the copolymers of acrylic acid and of acrylic esters such as the acrylic acid/ethyl acrylate/N-tert-butylacrylamide terpolymers sold, for example, under the name ULTRAHOLD® STRONG by the company BASF, copolymers derived from crotonic acid such as the vinyl acetate/vinyl tert-butylbenzoate/crotonic acid terpolymers such as MEXOMERE PW proposed by Chimex and the crotonic acid/vinyl acetate/vinyl neododecanoate terpolymers sold, for example, under the name Resin 28-29-30 by the company NATIONAL STARCH, the polymers derived from maleic, fumaric, itaconic acids or anhydrides with vinyl esters, vinyl ethers, vinyl halides, phenylvinyl derivatives, acrylic acid and its esters such as the monoesterified methylvinylether/maleic anhydride copolymers sold for example under the name GANTREZ® by the company ISP, the copolymers of methacrylic acid and methyl methacrylate sold under the name EUDRAGIT® L by the company RÖHM PHARMA, the copolymers of methacrylic acid and ethyl acrylate sold under the name LUVIMER® MAEX or MAE by the company BASF and the vinyl acetate/crotonic acid copolymers sold under the name LUVISET CA 66 by the company BASF and the vinyl acetate/crotonic acid copolymers grafted with polyethylene glycol sold under the name ARISTOFLEX® A by the company BASF, and the polymer sold under the name Fixate™ G-100 Polymer by the company Lubrizol.

Among the anionic fixing polymers mentioned above, within the scope of the present disclosure, further non-limiting mention can be made of the monoesterified methylvinyl ether/maleic anhydride copolymers sold under the name GANTREZ® ES 425 by the company ISP, the acrylic acid/ethyl acrylate/N-tert-butylacrylamide terpolymers sold under the name ULTRAHOLD® STRONG by the company BASF, the copolymers of methacrylic acid and methyl methacrylate sold under the name EUDRAGIT® L by the company RÖHM PHARMA, the vinyl acetate/vinyl tert-butylbenzoate/crotonic acid terpolymers and the crotonic acid/vinyl acetate/vinyl neododecanoate terpolymers sold under the name Resin 28-29-30 by the company NATIONAL STARCH, the copolymers of methacrylic acid and ethyl acrylate sold under the name LUVIMER® MAEX or MAE by the company BASF, the vinyl pyrrolidone/acrylic acid/lauryl methacrylate terpolymers sold under the name ACRYLIDONE® LM by the company ISP, the polymer sold under the name Fixate™ G-100 Polymer by the company Lubrizol.

The amphoteric fixing polymers that can be used according to the disclosure can be chosen from the polymers having units B and C randomly distributed in the polymer chain, where B denotes a unit derived from a monomer having at least one basic nitrogen atom and C denotes a unit derived from an acidic monomer having at least one carboxyl or sulphonic group, or else B and C can denote groups derived from zwitterionic monomers of carboxybetaines or sulphobetaines;

B and C can also denote a cationic polymer chain having primary, secondary, tertiary or quaternary amine groups, in which at least one of the amine groups bears a carboxyl or sulphonic group joined via a hydrocarbon group, or else B and C form part of a chain of a polymer with an ethylene-α,β-dicarboxyl unit in which one of the carboxyl groups has been reacted with a polyamine bearing at least one primary or secondary amine group.

The exemplary amphoteric fixing polymers corresponding to the definition given above are chosen from the following polymers:

(1) the copolymers with acidic vinyl units and with basic vinyl units, such as those resulting from the copolymerization of a monomer derived from a vinylic compound bearing a carboxyl group such as acrylic acid, methacrylic acid, maleic acid, alpha-chloroacrylic acid, and of a basic monomer derived from a substituted vinylic compound containing at least one basic atom, such as dialkylaminoalkyl methacrylate and acrylate, dialkylamino-alkylmethacrylamide and acrylamide. Such compounds may be described in U.S. Pat. No. 3,836,537.

(2) the polymers having units derived from :

a) at least one monomer chosen from the acrylamides and the methacrylamides substituted on the nitrogen atom with an alkyl group,

b) at least one acidic comonomer containing at least one reactive carboxyl group, and

c) at least one basic comonomer such as esters with primary, secondary, tertiary and quaternary amine substituents of acrylic and methacrylic acids, and the product of quaternization of dimethylaminoethyl methacrylate with dimethyl or diethyl sulphate.

The N-substituted acrylamides or methacrylamides according to the invention include but are not limited to the compounds in which the alkyl groups have from 2 to 12 carbon atoms, such as N-ethylacrylamide, N-tert-butylacrylamide, N-tert-octylacrylamide, N-octylacrylamide, N-decylacrylamide, N-dodecylacrylamide as well as the corresponding methacrylamides.

The acidic comonomers are chosen, for example, from acrylic, methacrylic, crotonic, itaconic, maleic, fumaric acids and the alkyl monoesters having from 1 to 4 carbon atoms of maleic and fumaric acids and anhydrides.

The basic comonomers, for example, are aminoethyl, butylaminoethyl, N,N′-dimethylaminoethyl, and N-tert-butylaminoethyl methacrylates.

In some embodiments, the copolymers are used whose CTFA designation (4th Ed., 1991) is octylacrylamide/acrylates/butylaminoethyl-methacrylate copolymer, such as the products sold under the name AMPHOMER® or LOVOCRYL® 47 by the company NATIONAL STARCH.

(3) the crosslinked polyaminoamides acylated partially or completely, derived from polyaminoamides of general formula:

CO—R₁₀—CO—Z (II)

in which R₁₀ represents a divalent group derived from a saturated dicarboxylic acid, from an aliphatic mono- or dicarboxylic acid with ethylenic double bond, from an ester of a lower alkanol having from 1 to 6 carbon atoms of these acids or from a group derived from the addition of any one of the acids with a bis-primary or bis-secondary amine, and Z denotes a group derived from a bis-primary, mono- or bis-secondary polyalkylene-polyamine and, for example, represents:

a) in the proportions from 60 to 100 mol. %, the group

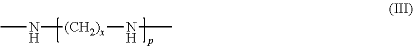

where x=2 and p=2 or 3, or else x=3 and p=2

this group being derived from diethylenetriamine, triethylenetetramine or dipropylenetriamine;

b) in the proportions from 0 to 40 mol. %, the above group (III), in which x=2 and p=1 and is derived from ethylenediamine, or the group derived from piperazine:

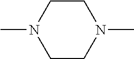

c) in the proportions from 0 to 20 mol. %, the group —NH—(CH₂)₆—NH— derived from hexamethylenediamine,

these polyaminoamides being crosslinked by a reaction of addition of a bifunctional crosslinking agent chosen from the epihalohydrins, diepoxides, dianhydrides, and bis-unsaturated derivatives, using 0.025 to 0.35 mol of crosslinking agent per amine group of the polyaminoamide, and acylated by the action of acrylic acid, chloroacetic acid and an alkane-sultone and salts thereof.

The saturated carboxylic acids are, for example, chosen from the acids having from 6 to 10 carbon atoms such as adipic, trimethyl-2,2,4-adipic and trimethyl-2,4,4-adipic, terephthalic acids, and acids with an ethylenic double bond, for example, acrylic, methacrylic, and itaconic acids.

The alkane-sultones used in the acylation are, for example, propane- or butane-sultone, the salts of the acylating agents are, for example, the sodium or potassium salts.

(4) the polymers having zwitterionic units of formula:

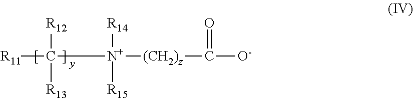

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, a methyl, ethyl or propyl group, R₁₄ and R₁₅ represent a hydrogen atom or an alkyl group in such a way that the sum of the carbon atoms in R₁₄ and R₁₅ does not exceed 10.

The polymers containing such units can also have units derived from non-zwitterionic monomers such as dimethyl- or diethylaminoethyl acrylate or methacrylate or alkyl acrylates or methacrylates, acrylamides or methacrylamides, or vinyl acetate.

Non-limiting examples include the methyl methacrylate/methyl dimethyl-carboxymethylammonio-ethylmethacrylate copolymers, such as the product sold under the name DIAFORMER Z301 by the company SANDOZ.

(5) the polymers derived from chitosan having monomer units corresponding to the following formulae:

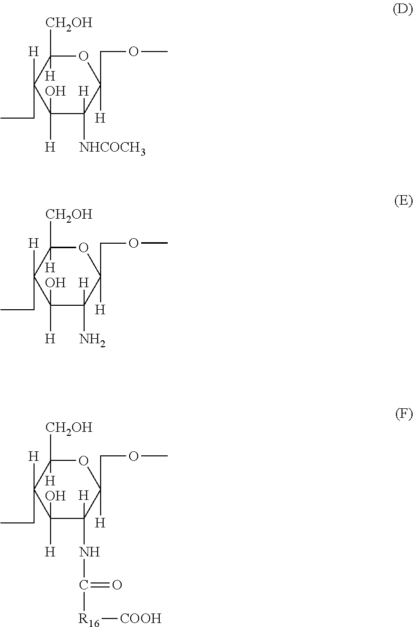

unit (D) being present in proportions ranging from 0 to 30%, unit (E) in proportions ranging from 5 to 50% and unit (F) in proportions ranging from 30 to 90%, it being understood that in this unit (F), R₁₆ represents a group of formula:

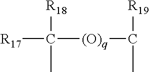

in which, if q=0, R₁₇, R₁₈ and R₁₉, which may be identical or different, each represent a hydrogen atom, a methyl, hydroxyl, acetoxy or amino residue, a monoalkylamine residue or a dialkylamine residue optionally interrupted by one or more nitrogen atoms and/or optionally substituted with at least one amine, hydroxyl, carboxyl, alkylthio, sulphonic groups, an alkylthio residue in which the alkyl group bears an amino residue, at least one of the groups R₁₇, R₁₈ and R₁₉ being in this case a hydrogen atom;

or if q=1, R₁₇, R₁₈ and R₁₉ each represent a hydrogen atom, as well as the salts formed by these compounds with bases or acids.

(6) The polymers with the units corresponding to general formula (V) are, for example, described in French Patent No. 1 400 366:

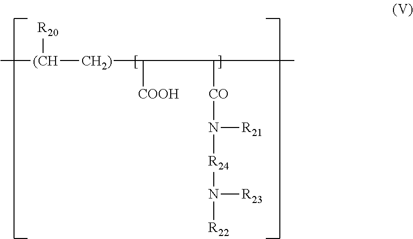

in which R₂₀ represents a hydrogen atom, a CH₃O, CH₃CH₂O, phenyl group, R₂₁ denotes a hydrogen atom or a lower alkyl group such as methyl, ethyl, R₂₂ denotes a hydrogen atom or a C₁-C₆ lower alkyl group such as methyl, ethyl, R₂₃ denotes a C₁-C₆ lower alkyl group such as methyl, ethyl or a group corresponding to the formula: —R₂₄—N(R₂₂)₂, R₂₄ representing a group —CH₂—CH₂—, —CH₂—CH₂—CH₂—, —CH₂—CH(CH₃)—, R₂₂ having the meanings given above.

(7) The polymers derived from the N-carboxyalkylation of chitosan such as N-carboxymethyl-chitosan or N-carboxybutyl-chitosan, sold under the name “EVALSAN” by the company JAN DECKER.

(8) The amphoteric polymers of the type -D-X-D-X chosen from:

a) the polymers obtained by the action of chloroacetic acid or sodium chloroacetate on compounds having at least one unit of formula:

-D-X-D-X-D- (VI)

where D denotes a group

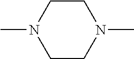

and X denotes the symbol E or E′, and E or E′, which may be identical or different, denote a divalent group, which is a linear or branched alkylene group, with up to 7 carbon atoms in the main chain, unsubstituted or substituted with hydroxyl groups and which can additionally contain oxygen, nitrogen, or sulphur atoms, 1 to 3 aromatic and/or heterocyclic rings; the oxygen, nitrogen and sulphur atoms being present in the form of ether, thioether, sulphoxide, sulphone, sulphonium, alkylamine, alkenylamine groups; hydroxyl, benzylamine, amine oxide, quaternary ammonium, amide, imide, alcohol, ester and/or urethane groups.

b) The polymers of formula:

-D-X-D-X— (VII)

where D denotes a group

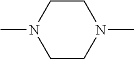

and X denotes the symbol E or E′ and at least once E′; E having the meaning stated above and E′ is a divalent group, which is a linear or branched alkylene group, with up to 7 carbon atoms in the main chain, unsubstituted or substituted with one or more hydroxyl groups and bearing one or more nitrogen atoms, the nitrogen atom being substituted with an alkyl chain interrupted optionally by an oxygen atom and which must have one or more carboxyl functions or one or more hydroxyl functions, betainized by reaction with chloroacetic acid or sodium chloroacetate.

(9) the alkyl(C₁-C₅)vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also contain other vinyl comonomers such as vinyl caprolactam.

Among the amphoteric fixing polymers mentioned above, non-limiting mention can be made of family (3) such as the copolymers whose CTFA designation is octylacrylamide/acrylates/butylamino-ethylmethacrylate copolymer, such as the products sold under the names AMPHOMER®, AMPHOMER® LV 71 or LOVOCRYL® 47 by the company NATIONAL STARCH and those of family (4) such as the methyl methacrylate/methyl dimethyl-carboxymethylammonio-ethylmethacrylate copolymers sold for example under the name DIAFORMER Z301 by the company SANDOZ.

The non-ionic fixing polymers for use according to the present disclosure are chosen, for example, from:

the polyalkyloxazolines;

the homopolymers of vinyl acetate;

the copolymers of vinyl acetate such as, for example, copolymers of vinyl acetate and acrylic ester, copolymers of vinyl acetate and ethylene, and copolymers of vinyl acetate and maleic ester, for example, dibutyl maleate;

the homopolymers and copolymers of acrylic esters such as, for example, the copolymers of alkyl acrylates and of alkyl methacrylates such as the products offered by the company RÖHM & HAAS under the names PRIMAL® AC-261 K and EUDRAGIT® NE 30 D, by the company BASF under the name 8845, by the company HOECHST under the name APPRETAN® N9212;

the copolymers of acrylonitrile and of a non-ionic monomer chosen for example from butadiene and alkyl (meth)acrylates; non-limiting examples include the products offered under the name CJ 0601 B by the company RÖHM & HAAS;

the homopolymers of styrene;

the copolymers of styrene, for example the copolymers of styrene and alkyl (meth)acrylate such as the products MOWILITH® LDM 6911, MOWILITH® DM 611 and MOWILITH® LDM 6070 offered by the company HOECHST, the products RHODOPAS® SD 215 and RHODOPAS® DS 910 offered by the company RHONE POULENC; the copolymers of styrene, of alkyl methacrylate and of alkyl acrylate; the copolymers of styrene and butadiene; and the copolymers of styrene, butadiene and vinylpyridine;

the polyamides;

the homopolymers of vinyl lactam such as the homopolymers of vinyl pyrrolidone and such as the polyvinyl caprolactam marketed under the name Luviscol® PLUS by the company BASF; and

the copolymers of vinyl lactam such as a poly(vinyl pyrrolidone/vinyl lactam) copolymer sold under the trade name Luvitec® VPC 55K65W by the company BASF, the poly(vinyl pyrrolidone/vinyl acetate) copolymers such as those marketed under the name PVPVA® S630L by the company ISP, Luviscol® VA 73, VA 64, VA 55, VA 37 and VA 28 by the company BASF; and the poly(vinyl pyrrolidone/vinyl acetate/vinyl propionate) terpolymers, for example that marketed under the name Luviscol® VAP 343 by the company BASF.

The alkyl groups of the non-ionic polymers mentioned above, for example, have from 1 to 6 carbon atoms.

According to the disclosure, non-limiting fixing polymers include the grafted silicone type, comprising a polysiloxane part and a part constituted of a non-silicone organic chain, one of the two parts constituting the main chain of the polymer and the other being grafted onto the main chain.

These polymers may be for example as described in European Patent Application Nos. EP 0 412 704 A, EPO 412 707 A, EP 0 640 105 A and EP 0 582 152 A, PCT Application Publication Nos. WO 95/00578 and WO 93/23009, and the U.S. Pat. No. 4,693,935, U.S. Pat. No. 4,728,571 and U.S. Pat. No. 4,972,037.

These polymers can be amphoteric, anionic or non-ionic, and they are, in some embodiments, anionic or non-ionic.

Such polymers are, for example, the copolymers that are obtainable by radical polymerization starting from a mixture of monomers comprising:

a) from 50 to 90 wt. % of tert-butyl acrylate,

b) from 0 to 40 wt. % of acrylic acid,

c) from 5 to 40 wt. % of a silicone macromer of formula

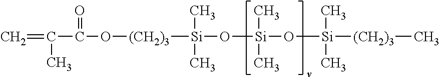

where v is a number from 5 to 700, the percentages by weight being calculated relative to the total weight of the monomers.

Other examples of grafted silicone polymers are, for example, polydimethylsiloxanes (PDMS) onto which mixed polymer units of the poly(meth)acrylic acid type and of the poly(alkyl (meth)acrylate) type are grafted via a joining group of the thiopropylene type, and polydimethylsiloxanes (PDMS) onto which polymer units of the poly(isobutyl(meth)acrylate) type are grafted via a joining group of the thiopropylene type.

As another type of silicone fixing polymers, non-limiting mention can be made of the product Luviflex® Silk marketed by the company BASF.

Polyurethanes different from the film-forming elastomeric, anionic polyurethane (A), functionalized or unfunctionalized, siliconized or unsiliconized, cationic, non-ionic, anionic or amphoteric, or mixtures thereof, can also be used as fixing polymers.

The polyurethanes to which the present disclosure relates, for example, may be those described in European Patent Application Nos. EP 0 751 162, EP 0 637 600, EP 0 648 485 and French Patent Application No. FR 2 743 297 of which the applicant is the proprietor, as well as in European Patent Application No EP 0 656 021 and PCT Application Publication No. WO 94/03510 of the company BASF, and European Patent Application No. EP 0 619 111 of the company National Starch.

For polyurethanes that can be used in the present disclosure, non-limiting mention can be made of the products marketed under the names LUVISET PUR® and LUVISET® Si PUR by the company BASF.

The individual concentration of additional fixing polymer(s) used in the compositions according to the present disclosure can be from 0.1 to 20%, such as from 0.5 to 10 wt. % relative to the total weight of the composition.

### Cosmetic Additive

The cosmetic composition, comprises water and/or at least one organic solvent, for example, chosen from the C₂-C₄ alcohols, such as ethanol, the polyols and ethers of polyols such as propylene glycols, polyethylene glycols, glycerol, acetone, propylene carbonate and benzyl alcohol.

The compositions according to the disclosure can also contain other cosmetically acceptable additives, for example non ionic, cationic, anionic or amphoteric thickeners, penetrating agents, perfumes, colorants, plasticizers, buffers, ceramides, pseudoceramides, vitamins or provitamins such as panthenol, opacifiers, reducing agents, emulsifiers, preservatives, mineral fillers, nacres, glitters, sunscreens, proteins, moisterizing agents, emollients, softening agents, antifoaming agents, antiperspirants, anti-free-radical agents, bactericides, chelating agents, anti-dandruff agents, antioxidants, alkalizing agents, acidifying agents, and any other additive conventionally used in cosmetic compositions intended to be applied on the hair.

### Cosmetic Composition

A person skilled in the art would be able to add the additives without disturbing the properties of the compositions of the disclosure.

In some embodiments, if the composition is an aqueous composition, the pH of the formulas associated with the disclosure is from 1 to 13, such as from 3 to 11, and further such as from 3 to 9 and more further such as from 5 to 7.

The composition according to the disclosure is, for example, used in non-rinse application on the hair.

Also provided is a method of shaping of the hair, comprising the application of a cosmetic composition according to the disclosure. For example, provided is a method of styling comprising the application of a composition according to the disclosure on the hair, optional rinsing off the hair, then shaping and drying of the hair.

The examples given below are intended to illustrate the disclosure without however limiting the scope thereof.

### EXAMPLES

In the following tables, the values were expressed in percentages by weight of active substance in the final formula.

The film-forming elastomeric, anionic polyurethanes (A) used in the examples were defined below on the basis of their chemical composition and mechanical properties. The compositions of the film-forming elastomeric, anionic polyurethanes (A) were expressed in dry matter.

| | Elongation | | Recovery |
|---|---|---|---|
| | at break | Instantaneous | at 300 s |
| | (%) | recovery (%) | (%) |
| A1 | 1300 | 70.5 | 90.6 |
| A2 | 833 | 74.9 | 90.8 |
| A3 | >1200 | 84.2 | 94.2 |
| A4 | | 77.6 | 93.5 |
| A5 | | 76.7 | 94.6 |
| A6 | 877 | 74.7 | 91.8 |

| | IPDI | PEO | PPG | DMPA | TMP | HNA | PDMS | MPEG | EDA | TEA | NaOH | TOTAL |
|---|---|---|---|---|---|---|---|---|---|---|---|---|
| A1 | 28.1 | 22.7 | 26.3 | 3.0 | 0.5 | 14.5 | | | 2.7 | 2.3 | | 100.0 |
| A2 | 28.9 | 16.7 | 23.0 | 2.9 | 0.5 | 23.0 | | | 2.7 | 2.1 | | 100.0 |
| A3 | 25.9 | 16.7 | 48.5 | 3.0 | | | | | 3.2 | 2.7 | | 100.0 |
| A4 | 23.8 | 15.3 | 44.4 | 2.8 | | | | 8.3 | 2.9 | 2.5 | | 100.0 |
| A5 | 24.6 | 15.9 | 46.4 | 2.9 | 1.6 | | 3.0 | | 3.0 | 0.9 | 1.6 | 100.0 |
| A6 | 29.1 | 14.1 | 49.7 | 2.9 | 0.5 | | | | 2.3 | 1.1 | 0.5 | 100.0 |

For all the polyurethanes A1 to A6, the ethylene oxide side chains had a molecular weight of 1200 g/mol and they represented more than 12 wt. % of the polyurethane (A).

### IPDI: Isophorone Diisocyanate

HNA: Hexane Neopentyl adipate polyester polyol (Mw=1000)
PPG: Polypropylene glycol (Mw=2000)
PEO: Polyethylene oxide-1,3-diol (Mw=1200)
DMPA: Dimethylolpropionic acid

### TMP: Trimethylolpropane

### EDA: Ethylenediamine

### TEA: Triethylamine

PDMS: Polydimethylsiloxane polyol

### MPEG: Methoxy Polyethylene Glycol

NaOH: Sodium hydroxide

### EtOH: Ethanol

The cationic fixing film forming polymers used in the examples were:
Polymère 1: SOFTCAT POLYMER SL-100 (Amerchol)
Polymère 2: STYLEZE W20 (ISP)
Polymère 3: CARBOPOL AQUA CC (Lubrizol)

| Compositions | 1 | 2 | 3 | 4 | 5 | 6 | 7 | 8 | 9 | 10 | 11 |
|---|---|---|---|---|---|---|---|---|---|---|---|
| As of the invention | 10 | 10 | 10 | 7 | 7 | 7 | 7 | 7 | 5 | 7 | 7 |
| (A1 or A2 or A3 or A4 or A5 | | | | | | | | | | | |
| or A6) | | | | | | | | | | | |
| Polymer 1: SOFTCAT | 1.5 | 1 | — | — | — | — | — | — | — | — | — |
| POLYMER SL-100 | | | | | | | | | | | |
| Polymer 2: | | | | | | | 5 | 10 | | | |
| STYLEZE W20 | | | | | | | | | | | |
| Polymer 3: | | | | | | | | | 7.5 | 10 | 15 |
| CARBOPOL AQUA CC | | | | | | | | | | | |
| Lactic acid | 1 | 1 | 0 | 2 | 2 | 2 | 0 | 0 | 1 | 2 | 2 |
| Ethanol | 2 | 2 | 2 | 2 | 2 | 2 | 2 | 2 | 2 | 2 | 2 |
| Distilled Water | Qsp | Qsp | Qsp | Qsp | Qsp | Qsp | Qsp | Qsp | Qsp | Qsp | Qsp |
| | 100 | 100 | 100 | 100 | 100 | 100 | 100 | 100 | 100 | 100 | 100 |

### Concentrations are Given in Weigh Percent Relative to the Total Weigh of the Composition.

### Protocol for Application of the Compositions at Room Temperature

Locks of natural brown hair were used; 2.7 g;.

1. Carried out standard shampoo,
2. Rinsed off the hair with water,
3. Dried the hair with a towel,
4. Applied the composition on the moist lock of hair with a bath ratio of 0.5,
5. Left to dry in the open air or a hair-dryer.

### Results

For each of the compositions according to the disclosure containing a film-forming elastomeric, anionic polyurethane (A) and a cationic fixing film forming polymer, the product flowed well in the hands and easily applied on the hair, so as to obtain a uniform distribution on hair.

During application and after drying, there was significant long lasting styling of hair.

The hair fixing was elastic and flexible. The polymer film formed was non-brittle.

What is claimed is:

1. A cosmetic composition comprising at least one film-forming elastomeric, anionic polyurethane (A), wherein the at least one film-forming elastomeric, anionic polyurethane (A) comprises:
(i) at least one side chain comprising at least one ethylene oxide unit, wherein the at least one side chain
represents from 12 to 80% by weight of the polyurethane (A)
has a Mw ranging from 1000 g/mol to 30,000 g/mol
(ii) one main chain comprising at least one unit derived from:
a polypropylene glycol (PPG) and optionally a second non-ionic polyol,
a dihydroxy-carboxylic acid and
at least one di-isocyanate, and optionally
a chain extender organic polyamine having an average of at least two primary amine groups,
And at least one cationic fixing film forming polymer.

2. The composition according to claim 1, wherein the at least one film-forming elastomeric, anionic polyurethane (A) is chosen such that the film obtained by drying of this polyurethane (A), at room temperature (24° C.±2° C.) and at a relative humidity of 48%±5%, presents a mechanical profile defined by at least:
(a) an elongation at break (ε_{b}) greater than or equal to 730%±5%; and/or
(b) an instantaneous recovery (Rᵢ) greater than or equal to 70%±5%, after elongation of 150%; and/or
(c) a recovery (R₃₀₀) at 300 seconds greater than or equal to 80%±5%.
and wherein the at least one film-forming elastomeric, anionic polyurethane (A) is soluble or dispersible, either in water or in a mixture of water/ethanol solvents comprising at most 30% by weight of ethanol.

3. The composition according to claim 2, wherein the at least one film-forming elastomeric, anionic polyurethane (A) is present at a concentration ranging from 0.01 to 20 wt. % relative to the total weight of the composition.

4. The composition according to claim 3, wherein the at least one film-forming elastomeric, anionic polyurethane (A) is present at a concentration ranging from 1 to 10 wt. % relative to the total weight of the composition.

5. The composition according to claim 2, further comprising at least one α-hydroxy or α-keto acid.

6. The composition according to claim 5, comprising at least one α-hydroxy acid preferably chosen among lactic acid, malic acid, citric acid, gluconic acid, and tartaric acid.

7. The composition according to claim 5, wherein α-hydroxy or α-keto acids represent from 0.1 to 10% wt relative to the total weight of the composition.

8. The composition according to claim 2, further comprising at least one surfactant chosen from anionic, non-ionic, amphoteric and cationic surfactants.

9. The composition according to claim 2, further comprising at least one silicone.

10. The composition according to claim 9, wherein the at least one silicone is chosen from volatile and non-volatile silicones, cyclic, linear and branched, unmodified and modified with organic groups.

11. The composition according to claim 2, further comprising at least one non-silicone fatty substance.

12. The composition according to claim 10, wherein the at least one non-silicone fatty substance is chosen from mineral, vegetable, animal and synthetic oils; waxes, fatty esters, ethoxylated and non-ethoxylated fatty alcohols, and fatty acids.

13. The composition according to claim 2, further comprising at least one fixing polymer chosen from anionic, amphoteric and non-ionic fixing polymers, different from the polyurethanes (A).

14. The composition according to claim 2, comprising water and/or at least one organic solvent chosen from the C₂ to C₄ alcohols, the polyols, the polyol ethers, acetone, propylene carbonate and benzyl alcohol.

15. The composition according to claim 2, further comprising at least one additive chosen from non ionic, cationic, anionic or amphoteric thickeners, penetrating agents, perfumes, colorants, plasticizers, buffers, ceramides, pseudoceramides, vitamins or provitamins such as panthenol, opacifiers, reducing agents, emulsifiers, preservatives, mineral fillers, nacres, glitters, sunscreens, proteins, moisterizing agents, emollients, softening agents, antifoaming agents, antiperspirants, anti-free-radical agents, bactericides, chelating agents, anti-dandruff agents, antioxidants, alkalizing agents, acidifying agents, and other additives conventionally used in cosmetic compositions intended to be applied on the hair.

16. The composition according to claim 2, wherein the composition is a non-rinse composition.

17. A cosmetic method for styling the hair, comprising: applying to the hair a cosmetic composition, wherein the cosmetic composition comprises at least one film-forming elastomeric, anionic polyurethane (A), wherein the at least one film-forming elastomeric, anionic polyurethane (A) comprises:
(i) at least one side chain comprising at least one ethylene oxide unit, wherein the at least one side chain
represents from 12 to 80% by weight of the polyurethane (A)
has a Mw ranging from 1000 g/mol to 30,000 g/mol
(ii) one main chain comprising at least one unit derived from:
a polypropylene glycol (PPG) and optionally a second non-ionic polyol,
a dihydroxy-carboxylic acid and
at least one di-isocyanate, and optionally
a chain extender organic polyamine having an average of at least two primary amine groups,
and at least one cationic fixing film forming polymer.

18. A cosmetic method according to claim 17, and wherein the at least one film-forming elastomeric, anionic polyurethane (A) is chosen such that the film obtained by drying at room temperature (24° C.±2° C.) and at a relative humidity of 48%±5%, presents a mechanical profile defined by at least:
(a) an elongation at break (ε_{b}) greater than or equal to 730%, ±5% (I.E. 694%-766%); and/or
(b) an instantaneous recovery (Rᵢ) greater than or equal to 70%±5%, after elongation of 150%; and/or
(c) a recovery (R₃₀₀) at 300 seconds greater than or equal to 80%, ±5%.
the polyurethane (A) being soluble or dispersible, either in water, or in a mixture of water/ethanol solvents comprising at most 30% by weight of ethanol.
